(19) United States
(12) Patent Application Publication
(10) Pub. No.: US 2026/0158005 A1
(43) Pub. Date: Jun. 11, 2026

(54) A SALT-BASED ANTIFUNGAL POWDER PLATFORM FORMULATION FOR AEROSOLIZATION

(71) Applicants: AGENCY FOR SCIENCE, TECHNOLOGY AND RESEARCH, Singapore (SG); NATIONAL UNIVERSITY HOSPITAL (SINGAPORE) PTE LTD, Singapore (SG)

(72) Inventors: Wen Chien Desmond HENG, Singapore (SG); Say Kong NG, Singapore (SG); Sie Huey LEE, Singapore (SG); Woon Pei Jeanette TEO, Singapore (SG)

(21) Appl. No.: 18/707,341

(22) PCT Filed: Oct. 17, 2022
(86) PCT No.: PCT/SG2022/050738

§ 371 Date: May 3, 2024

(30) Foreign Application Priority Data
Nov. 9, 2021 (SG) 10202112434X

(51) Int. Cl. A61K 31/4196; A61K 9/00; A61K 31/496; A61K 31/506; A61K 47/02; A61K 47/18; A61P 31/10

(52) U.S. Cl. CPC A61K 31/4196; A61K 9/0075; A61K 31/496; A61K 31/506; A61K 47/02; A61K 47/183; A61P 31/10

(57) ABSTRACT

The present invention relates to a salt-based antifungal powder platform formulation comprising a sodium salt and L-leucine as excipients to be formulated alongside an antifungal agent to obtain a salt-based antifungal powder formulation, wherein the sodium salt is at a concentration of 5% w/w or less and the L-leucine is at a concentration of 30% w/w.

### FIELD OF THE INVENTION

The present invention relates to a salt-based antifungal powder platform formulation for aerosolization. In particular, the invention relates to a salt-based antifungal powder platform formulation to be formulated alongside an antifungal agent for high aerosol performance and efficacy.

### BACKGROUND

Pulmonary fungal infection is a severe clinical problem, especially in immunocompromised patients including those suffering from immunodeficiency disorders such as HIV/Aids and cancer patients who undergo chemotherapy, as well as those patients who undergo organ transplant. Most commercial antifungal preparations for lung infections are administered by oral or intravenous mode of administration. Such mode of administration are fraught with systemic toxicity, side effects and limited availability of the therapeutic drug in the lungs as the drug is not targeted at the lungs when the drug is administered.

It is therefore desirable to provide a more efficacious antifungal preparation that directly targets the lungs and confers high aerosol performance and efficacy.

### SUMMARY OF INVENTION

In accordance with a first aspect of this invention, there is provided a salt-based antifungal powder platform formulation. The platform formulation comprising a sodium salt and L-leucine as excipients to be formulated alongside an antifungal agent to obtain a salt-based antifungal powder formulation, wherein the sodium salt is at a concentration of 5% w/w or less and the L-leucine is at a concentration of 30% w/w.

In some embodiments, the sodium salt is selected from the group consisting of sodium sulfate, sodium acetate, sodium bicarbonate, sodium butyrate, sodium chloride and sodium metabisulfite.

In some embodiments, the antifungal agent is selected from the group consisting of Fluconazole, Itraconazole or Voriconazole.

In accordance with a second aspect of this invention, a salt-based antifungal powder formulation comprising a sodium salt at a concentration of 5% w/w or less, L-leucine at a concentration of 30% w/w and an antifungal agent is provided.

In accordance with a third aspect of this invention, a delivery system comprising an inhaler and a salt-based antifungal powder formulation of the present invention is provided.

### BRIEF DESCRIPTION OF THE DRAWINGS

The above advantages and features of a formation in accordance with this invention are described in the following detailed description and are shown in the drawings:

FIG. 1 is a graph showing the signal-to-noise (S/N) ratio response curve for in vitro aerosol performance. Letters A to D represent the experimental parameters and corresponding levels.

FIG. 2 is a graph showing the S/N ratio response curve for antifungal activity. Letters A to D represent the experimental parameters and corresponding levels.

FIGS. 3(a) to (c) are the Field Emission Scanning Election Microscopic (FESEM) images of the samples (a) OP-A, (b) OP-B and (c) OP-C described in Example 2.

### DETAILED DESCRIPTION

The present invention relates to a salt-based antifungal powder platform formation that can be applied to a variety of antifungal agents, to obtain high aerosol performance and efficacy. Fungi may cause lung disease through direct infection of pulmonary tissue, through infection of pulmonary air spaces/lung cavities, or through their ability to trigger an immunological reaction when fungal material is inhaled. Fungal infections are of a particular concern especially for the immunocompromised patients who are at a higher risk of contracting the infections in the lung. The resulting salt-based antifungal powder formulation of the present invention can be prepared in the form of a direct lung-targeting solid-dose that can be delivered to the lung directly in an effective way to increase drug concentration in lung tissue to treat the fungal infections in the lung.

In one aspect or embodiment, the salt-based antifungal powder platform formulation comprises a sodium salt and L-leucine as excipients to be formulated alongside an antifungal agent to obtain a salt-based antifungal powder formulation, wherein the sodium salt is at a concentration of 5% w/w or less and the L-leucine is at a concentration of 30% w/w.

The sodium salt is selected from the group consisting of sodium sulfate, sodium acetate, sodium bicarbonate, sodium butyrate, sodium chloride and sodium metabisulfite. In various embodiments, the sodium salt is sodium sulfate.

The salt-based antifungal powder platform formation can be applied to a variety of antifungal agents. As used herein, the term “antifungal agent” is intended to mean a substance capable of inhibiting or preventing the growth, viability and/or reproduction of fungi. The antifungal agent can be a broad spectrum antifungal agent or it can also be specific to one or more particular species of fungus. Examples of antifungal agents include, but are not limited to, azoles such as Fluconazole, Itraconazole, Voriconazole, Isavuconazole, Posaconazole, etc.

In a second aspect or embodiment, a salt-based antifungal powder formulation is provided. The formulation comprises a sodium salt at a concentration of 5% w/w or less, L-leucine at a concentration of 30% w/w and an antifungal agent.

In various embodiments, the antifungal agent is selected from the group consisting of Fluconazole, Itraconazole and Voriconazole.

The sodium salt is selected from the group consisting of sodium sulfate, sodium acetate, sodium bicarbonate, sodium butyrate, sodium chloride and sodium metabisulfite. In some embodiments, the sodium salt is sodium sulfate.

The salt-based antifungal powder formulation is a dry powder formulation for inhalation. The dry powder formulation is prepared by spray drying the formulation to obtain spray-dried particles. The spray-dried particles comprise fixed dose combinations of one or more antifungal agents that allows the solid dose to be delivered directly to the lung via dry powder inhalers for the treatment of fungal infections in the lung. This avoids excessive systemic exposures when treating the infected lung. This mode of delivery of the formulation (or drug) allows non-intravenous route of administration of the formulation or drug to patients and it helps to improve patient compliance.

In another aspect or embodiment, a delivery system comprising an inhaler and a salt-based antifungal powder formulation of the present invention is provided.

The salt-based antifungal powder platform formulation can be formulated with suitable antifungal agent to obtain a salt-based antifungal powder formulation for use in producing pharmaceutical compositions and products, animal feed (as antifungal feed additives) and powder fungicides for use in agriculture.

The salt-based platform formulation of the present invention has several advantages. One of the advantages is that the platform formulation can be readily adapted to a variety of antifungal agents. The platform formulation confers high aerosol performance and efficacy to the antifungal agents. It allows development of a more efficacious antifungal preparation that directly targets the lungs. The low salt content (5% w/w or less) of the platform formulation helps to minimize health risks such as hypertension. The platform formulation shows significant improvements over unformulated drugs (see Examples hereinbelow).

To facilitate a better understanding of the present invention, the following examples of specific embodiments are given. In no way should the following examples be read to limit or define the entire scope of the invention. One skilled in the art will recognize that the examples set out below are not an exhaustive list of the embodiments of this invention.

### EXAMPLES

### Example 1

Salts could potentially have antifungal activity. This example demonstrates how a salt-based antifungal powder platform formulation of the present invention is derived. An experimental design was used to screen several salts that could be used as adjuvants in the platform formulation. Four formulation parameters (factors) A, B, C and D were selected, and they are as illustrated in Table 1. Three generic antifungal agents (parameter C), namely Fluconazole, Itraconazole and Voriconazole were used as the model/test compounds.

TABLE 1
Experimental parameters (factors) and levels

| | Level | | | | | |
|---|---|---|---|---|---|---|
| Parameter | 1 | 2 | 3 | 4 | 5 | 6 |
| A. Excipient type (sodium salt) | sodium | sodium | sodium | sodium | sodium | sodium |
| | acetate | bicarbonate | [symbol] | chloride | metabisulfate | sulfate |
| B. Excipient concentration (% w/w) | 5 | 10 | 15 | — | — | — |
| C. Antifungal drug type | Fluconazole | Itraconazole | Voriconazole | — | — | — |
| D. L-leucine concentration (% w/w) | 10 | 20 | 30 | — | — | — |

[symbol]

indicates data missing or illegible when filed

The responses were Fine Particle Fraction (FPF) and Anti-fungal Activity (MIC—Minimum Inhibitory Concentration) (Table 2), while the tested pathogen was Candida albicans.

TABLE 2
Experimental parameters (factors) and levels

| | | | | | FPF (%) | | MIC (μg/ml) | |
|---|---|---|---|---|---|---|---|---|
| | | | | | | S/N | | S/N |
| | Parameter | | | | y mean | ratio | y mean | ratio |
| [symbol] | A | B | C | D | (n = 3) | (dB) | (n = 3) | (dB) |
| 1 | 1 | 1 | 1 | 1 | 38.4 ± [symbol] .9 | 31 | 0.17 ± 0.07 | 15 |
| 2 | 1 | 2 | 2 | 2 | [symbol] 1.1 ± 2. [symbol] | 30 | 0.1 [symbol] ± 0.11 | 16 |
| 3 | 1 | 3 | 3 | 3 | 32.9 ± 0.8 | 30 | 0.0 [symbol] ± 0.00 | 24 |
| 4 | 2 | 1 | 1 | 2 | 33.7 ± 2.6 | 31 | 0.2 [symbol] ± 0.00 | 12 |
| 5 | 2 | 2 | 2 | 3 | 30. [symbol] ± 1.0 | 30 | 0.13 ± 0.00 | 18 |
| 6 | 2 | 3 | 3 | 1 | 10.2 ± 1.0 | 20 | 0.25 ± 0.00 | 12 |
| 7 | 3 | 1 | 2 | 1 | 17.6 ± 1.1 | 2 [symbol] | 0.21 ± 0.07 | 13 |
| 8 | 3 | 2 | 3 | 2 | 17.2 ± 1.8 | 25 | 0.13 ± 0.00 | 18 |
| 9 | 3 | 3 | 1 | 3 | [symbol] . [symbol] ± 2. [symbol] | 32 | 0.17 ± 0.07 | 15 |
| 10 | 4 | 1 | 3 | 3 | 46.7 ± 1.3 | 33 | 0.04 ± 0.02 | 27 |
| 11 | 4 | 2 | 1 | 1 | [symbol] 1.2 ± 3.4 | 34 | 0.1 [symbol] ± 0.00 | 18 |
| 12 | 4 | 3 | 2 | 2 | 38.0 ± 0.4 | 32 | 0.13 ± 0.00 | 18 |
| 13 | 5 | 1 | 2 | 3 | 44.8 ± 3.4 | 33 | 0.04 ± 0.02 | 28 |
| 14 | 5 | 2 | 3 | 1 | 3 [symbol] .8 ± 3.1 | 31 | 0.10 ± 0.13 | 17 |
| 15 | 5 | 3 | 1 | 2 | 46.4 ± 0.9 | 33 | 0.13 ± 0.00 | 18 |
| 16 | 6 | 1 | 3 | 2 | 40.1 ± 1.5 | 32 | 0.0 [symbol] ± 0.06 | 23 |
| 17 | 6 | 2 | 1 | 3 | 58.6 ± 3.4 | 3 [symbol] | 0.1 [symbol] ± 0.00 | 18 |
| 18 | 6 | 3 | 2 | 1 | 40.8 ± 0. [symbol] | 32 | 0.0 [symbol] ± 0.06 | 23 |

y raw data ( [symbol] variable) [symbol]
indicates data missing or illegible when filed

### Fine Particle Fraction (FPF)

The FPF was influenced mainly by the type of salt excipient (i.e., parameter A; Rank 1), with sodium sulfate as one of the preferred options. The optimum results for all the parameters are: A6, B1, C1, D3.

TABLE 3
S/N response for fine particle fraction, FPF (dB)

| Level | A | B | C | D |
|---|---|---|---|---|
| 1 | 30.5 | 30.9 | 32.7 | 29.0 |
| 2 | 26.8 | 30.8 | 30.2 | 30.3 |
| 3 | 27.0 | 29.9 | 28.6 | 32.2 |
| 4 | 33.0 | | | |
| 5 | 32.4 | | | |
| 6 | 33.3 | | | |
| Range | 6.5 | 1.0 | 4.2 | 3.2 |
| Rank | 1 | 4 | 2 | 3 |

Letters (A-D) denote the experimental parameters

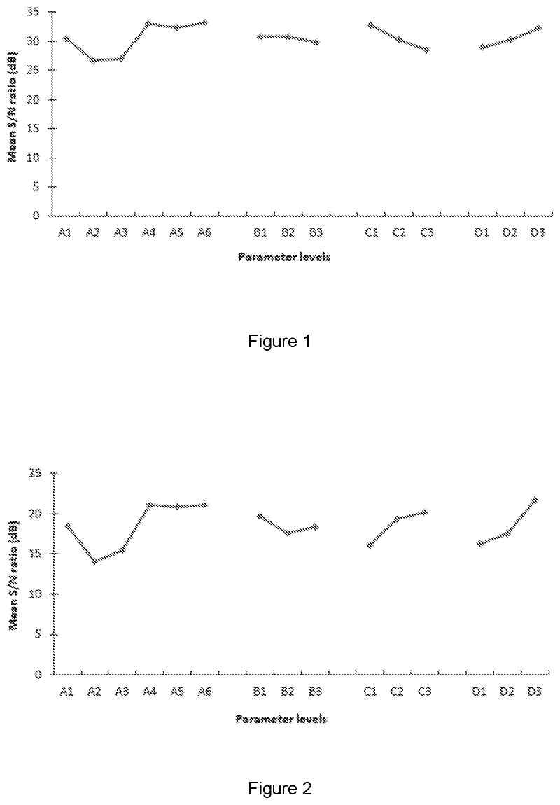

FIG. 1 shows the S/N ratio response curve for in-vitro aerosol performance. Letters A, B, C and D represent the experimental parameters and corresponding levels.

### Antifungal Activity

The antifungal activity was influenced mainly by the type of salt excipient as well (i.e. parameter A; Rank 1), with sodium sulfate as one of the preferred options. The optimum results for all the parameters are: A6, B1, C3, D3.

TABLE 4
S/N response for antifungal activity (dB)

| Level | A | B | C | D |
|---|---|---|---|---|
| 1 | 18.5 | 19.6 | 16.1 | 16.3 |
| 2 | 14.0 | 17.5 | 19.3 | 17.5 |
| 3 | 15.5 | 18.3 | 20.1 | 21.7 |
| 4 | 21.1 | | | |
| 5 | 20.8 | | | |
| 6 | 21.1 | | | |
| Range | 7.1 | 2.1 | 4.1 | 5.4 |
| Rank | 1 | 4 | 3 | 2 |

Letters (A-D) denote the experimental parameters

FIG. 2 shows the S/N ratio response curve for antifungal activity. Letters A, B, C, D represent the experimental parameters and corresponding levels.

### Platform Formulation

The optimum parameters based on FPF enhancement are A6, B1, C1, D3. The optimum parameters based on antifungal activity enhancement are A6, B1, C3, D3.

As parameter C is an antifungal agent, and parameters A, B and D are the excipients, a salt-based excipient platform for formulating with antifungal agent is derived (i.e., A6, B1, D3).

From the method described hereinabove, a salt-based antifungal powder platform formulation comprising (1) sodium sulfate at a concentration of 5% w/w; and (2) L-leucine at a concentration of 30% w/w is derived.

### Example 2

### Testing the Platform Formulation

The salt-based platform formulation described in Example 1 was prepared and formulated with each of the three generic antifungal agents: (A) Fluconazole, (B) Itraconazole, and (C) Voriconazole to test for its robustness.

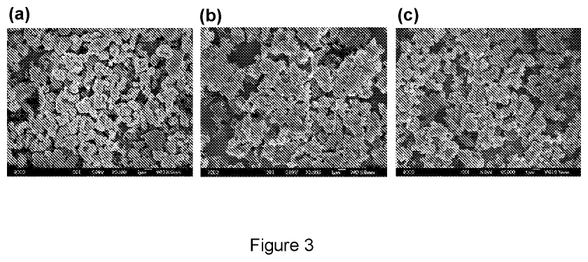

FIG. 3 are FESEM images of (a) OP-A, (b) OP-B and (c) OP-C. The images show that for all three test antifungal agents, the obtained particle size distributions were very narrow (i.e. uniform particles). The FPF and MIC readings for the three test antifungal agents are shown in Table 5.

TABLE 5
Performance of test antifungal agents
using the platform formulation

| | Parameter | | | | FPF | MIC |
|---|---|---|---|---|---|---|
| | A | B | C | D | (%) | (μg/ml) |
| Optimized Formulation | | | | | | |
| using Platform | | | | | | |
| OP-A | 6 | 1 | 1 | 3 | 56.2 ± 3.3 | 0.06 ± 0.00 |
| OP-B | 6 | 1 | 2 | 3 | 49.1 ± 1.3 | <0.03 ± 0.00 |
| OP-C | 6 | 1 | 2 | 3 | 55.2 ± 1.0 | <0.03 ± 0.00 |
| Unformulated | | | | | | |
| A | — | — | 1 | — | 2.6 ± 0.1 | 0.25 ± 0.00 |
| B | — | — | 2 | — | 3.4 ± 0.5 | <0.03 ± 0.00 |
| C | — | — | 3 | — | 3.5 ± 0.3 | <0.03 ± 0.00 |

*Sample OP-A and A: Fluconazole
*Sample OP-B and B: Itraconazole
*Sample OP-C and C: Voriconazole

From Table 5, we can see that when the three test antifungal agents were formulated using the platform technology (i.e., sodium sulfate at 5% w/w, L-leucine at 30% w/w, and spray dry), there was approximately 15 to 20 times improvement in the FPF over unformulated antifungal agents. Antifungal activity was either maintained or enhanced, with approximately 4 times improvement for fluconazole.

The above results show that the salt-based antifungal powder platform formulation of the present invention confers high aerodynamicity and activity to the antifungal agents.

Although embodiments of the present invention have been shown and described, the present invention is not limited to the described embodiments. Instead, it would be appreciated by those skilled in the art that changes may be made to the embodiments without departing from the scope of the invention, the scope of which is set forth in the following claims.

1. A salt-based antifungal powder platform formulation comprising a sodium salt and L-leucine as excipients to be formulated alongside an antifungal agent to obtain a salt-based antifungal powder formulation, wherein the sodium salt is at a concentration of 5% w/w or less and the L-leucine is at a concentration of 30% w/w.

2. The salt-based antifungal powder platform formulation according to claim 1, wherein the sodium salt is selected from the group consisting of sodium sulfate, sodium acetate, sodium bicarbonate, sodium butyrate, sodium chloride and sodium metabisulfite.

3. The salt-based antifungal powder platform formulation according to claim 2, wherein the sodium salt is sodium sulfate.

4. The salt-based antifungal powder platform formulation according to claim 1, wherein the antifungal agent is selected from the group consisting of Fluconazole, Itraconazole and Voriconazole.

5. The salt-based antifungal powder platform formulation according to claim 4, wherein the antifungal agent is Fluconazole.

6. The salt-based antifungal powder platform formulation according to claim 4, wherein the antifungal agent is Itraconazole.

7. The salt-based antifungal powder platform formulation according to claim 4, wherein the antifungal agent is Voriconazole.

8. A salt-based antifungal powder formulation comprising a sodium salt at a concentration of 5% w/w or less, L-leucine at a concentration of 30% w/w and an active agent, wherein the active agent is an antifungal agent.

9. The salt-based antifungal powder formulation according to claim 8, wherein the sodium salt is selected from the group consisting of sodium sulfate, sodium acetate, sodium bicarbonate, sodium butyrate, sodium chloride and sodium metabisulfite.

10. The salt-based antifungal powder formulation according to claim 9, wherein the sodium salt is sodium sulfate.

11. The salt-based antifungal powder formulation according to claim 1, wherein the antifungal agent is selected from the group consisting of Fluconazole, Itraconazole and Voriconazole.

12. The salt-based antifungal powder formulation according to claim 11, wherein the antifungal agent is Fluconazole.

13. The salt-based antifungal powder formulation according to claim 11, wherein the antifungal agent is Itraconazole.

14. The salt-based antifungal powder formulation according to claim 11, wherein the antifungal agent is Voriconazole.

15. A delivery system comprising an inhaler and a salt-based antifungal powder formulation according to claim 8.
